(19) United States
(12) Patent Application Publication
(10) Pub. No.: US 2022/0062020 A1
(43) Pub. Date: Mar. 3, 2022

(54) Lumbar Support Belt

(71) Applicant: James S. Striano, D.C., Yonkers, NY (US)

(72) Inventor: James S. Striano, D.C., Yonkers, NY (US)

(73) Assignee: James S. Striano, D.C., Yonkers, NY (US)

(21) Appl. No.: 17/007,749

(22) Filed: Aug. 31, 2020

(51) Int. Cl. A61F 5/02

(52) U.S. Cl. CPC A61F 5/028

(57) ABSTRACT

A lumbar support belt includes a posterior panel, and a first anterior panel coupled to the posterior panel by a first wing extending from the posterior panel. A second anterior panel is coupled to the posterior panel by a second wing extending from the posterior panel. First and second attachment portions are attached to the posterior panel by a first posterior strap and second posterior strap, respectively. The first posterior strap and second posterior strap are each being anchored to the posterior panel at two points. First and second anterior straps are each anchored to either one of the first and second anterior panel at two points and securable to the first and second attachment portions.

### BACKGROUND OF THE INVENTION

Belts constructed to at least partially surround the lower back region of the human torso are known for the treatment and rehabilitation of spinal dysfunction. The portions of such belts that contact the wearer are typically either flexible, permitting unintended motion by the wearer when the belt is bent, or inflexible, permitting unintended motion by the wearer to the extent that the inflexible portions do not press entirely onto the wearer's body. This unintended range of motion can, to some extent, be mitigated by proper fitting of the belt. Tightening the belt can disrupt placement of its features on the wearer because the straps used for tightening the belt are also responsible for holding the features of the belt in place prior to tightening.

### BRIEF SUMMARY OF THE INVENTION

In an aspect of the disclosure, a belt may include a posterior panel and two anterior panels connected to the posterior panel by two wings. The wings may be elastic, enabling flexibility in the placement of the panels on the wearer's abdomen and retention of the panels on the wearer's abdomen prior to any tightening. The two anterior panels may be securable to one another, such as by mutual adhesion, to enable the two anterior panels hold each other in an overlapping placement.

Two straps may be anchored onto the posterior plate, and another two straps may be anchored onto one of the anterior panels. The straps may be securable to one another to provide an adjustable tightness between the anterior panels and the posterior panel independent of tension on the wings. Each of the straps may have a Y shape, with one end anchored to the respective panel at a superior position, and another end anchored to the respective panel at an inferior position, to distribute load effectively across the panels and prevent separation of either panel from the wearer's abdomen at either the superior or inferior end. The posterior panel and one of the anterior panels may each include a pocket for receiving a rigid plate for further restricting movement of the wearer and distributing load across the wearer's abdomen. The plates may have differing arcuate shapes for matching typical abdominal anatomy and lordotic curve. The plate for the posterior panel may additionally include a rib running there along for extending between the wearer's spinal erectors to directly immobilize the spine.

In another aspect, a lumbar support belt may comprise, a posterior panel, a first anterior panel coupled to the posterior panel by a first wing extending from the posterior panel, a second anterior panel coupled to the posterior panel by a second wing extending from the posterior panel, first and second attachment portions attached to the posterior panel by a first posterior strap and second posterior strap, respectively, the first posterior strap and second posterior strap each being anchored to the posterior panel at two points, and first and second anterior straps each anchored to either one of the first and second anterior panel at two points and securable to the first and second attachment portions.

In some arrangements, the first and second anterior straps may each be anchored to the first anterior panel at a relatively superior point and a relatively inferior point.

In some arrangements, the first and second posterior straps may each be anchored to the posterior panel at a relatively superior point and a relatively inferior point.

In some arrangements, the first anterior strap and the second anterior strap may each have a Y shape, with first and second ends being anchored to the respective one of the first anterior panel and second anterior panel and a third end being free.

In some arrangements, the third ends of the first anterior strap and second anterior strap may be self-adhesible.

In some arrangements, the third ends of the first anterior strap and the second anterior strap may include hooks insertable through the first attachment portion and the second attachment portion.

In some arrangements, the first posterior strap and the second posterior strap may each have a Y shape, with first and second ends being anchored to the posterior panel and a third end being free, with the respective one of the first attachment portion and the second attachment portion being at the third end.

In some arrangements, the first wing and the second wing may each include an elastic segment enabling elongation of the respective one of the first wing and the second wing between the posterior panel and the respective one of the first anterior panel and second anterior panel.

In some arrangements, the posterior panel may include a pocket for a receiving a rigid posterior plate.

In some arrangements, the posterior plate may include a lumbar portion and a thoracic portion that is adjacent to the lumbar portion in a longitudinal direction and narrower than the lumbar portion in a lateral direction that is perpendicular to the longitudinal direction.

In some arrangements, the posterior plate may include a concave side, a convex side opposite the concave side, and a rib extending longitudinally along the convex side.

In some arrangements, one or both of the first anterior panel and second anterior panel may include a pocket for receiving the anterior plate.

In some arrangements, the first wing may terminate in the first anterior panel and the second wing terminates in the second anterior panel.

In some arrangements, the first and second anterior straps may each be securable to the respective first and second attachment portions at a user selectable position along the respective one of the first anterior strap and second anterior strap.

In some arrangements, the first and second anterior panel may be mutually adhesible.

In some arrangements, the first and second attachment portion may be a first and second ring secured to an end of the respective first or second posterior strap.

In another aspect, a lumbar support belt may comprise a posterior panel, first and second posterior straps each anchored to the posterior panel at two points, an anterior panel, and first and second anterior straps each anchored to the anterior panel at two points, the first and second anterior straps each being securable to a respective one of the first and second posterior straps such that, when secured, a combined length of the first anterior strap and first posterior strap is user selectable and a combined length of the second anterior strap and the second posterior strap is user selectable.

In some arrangements, the anterior panel may be coupled to the posterior panel by a wing extending from the posterior panel.

In some arrangements, the anterior panel may include a connecting panel adhesible to the anterior panel, the connecting panel being coupled to the posterior panel by a wing extending from the posterior panel.

In some arrangements, the first and second anterior straps may each be anchored to the first anterior panel at a relatively superior point and a relatively inferior point.

In some arrangements, the first and second posterior straps may each be anchored to the posterior panel at a relatively superior point and a relatively inferior point.

In some arrangements, either or both of the posterior panel and anterior panel may include a pocket for receiving a rigid plate.

In another aspect, a method of providing hydrostatic lift may include placing a posterior panel against a posterior of a wearer's abdomen, placing a first anterior panel against an anterior of the wearer's abdomen, and tightening the posterior panel and first anterior panel against the wearer's abdomen by adjusting a connection between a first posterior strap anchored at two points to the posterior panel and a first anterior strap anchored at two points to the anterior panel and a connection between a second posterior strap anchored at two points to the posterior panel and a second anterior strap anchored at two points to the anterior panel.

In some arrangements, the method may include placing a second anterior panel against the anterior of the wearer's abdomen before tightening the posterior panel and first anterior panel against the wearer's abdomen.

In some arrangements, placing the first anterior panel against the anterior of the wearer's abdomen may include placing the first anterior panel over the second anterior panel and establishing an adhesive connection between the first and second anterior panels.

In some arrangements, the method may include inserting a rigid posterior plate into the posterior panel before tightening the posterior panel and first anterior panel against the wearer's abdomen.

In some arrangements, the method may include inserting a rigid anterior plate into the anterior panel before tightening the posterior panel and first anterior panel against the wearer's abdomen.

In some arrangements, adjusting the connection between the first posterior strap and the first anterior strap and adjusting the connection between the second posterior strap and second anterior strap may include either pulling the first and second anterior straps through rings connected to the first and second posterior straps, or pulling the first and second posterior straps through rings connected to the first and second anterior straps.

### BRIEF DESCRIPTION OF THE DRAWINGS

FIG. 1 is a right side perspective view of a belt according to an aspect of the disclosure.

FIG. 2 is a perspective view of the belt of FIG. 1 in use as viewed from behind the user.

FIG. 3 is a left side perspective view of a belt according to another aspect of the disclosure.

FIG. 4 is a top plan simplified schematic view of the belt of FIG. 3 looped as when worn about a user's torso.

FIG. 5A is a front elevation view of a plate for insertion into an anterior panel of the belt of FIG. 1.

FIG. 5B is a top view of the plate of FIG. 5A.

FIG. 6A is a front elevation view of a plate for insertion into a posterior panel of the belt of FIG. 1.

FIG. 6B is a side elevation view of the plate of FIG. 6A.

### DETAILED DESCRIPTION

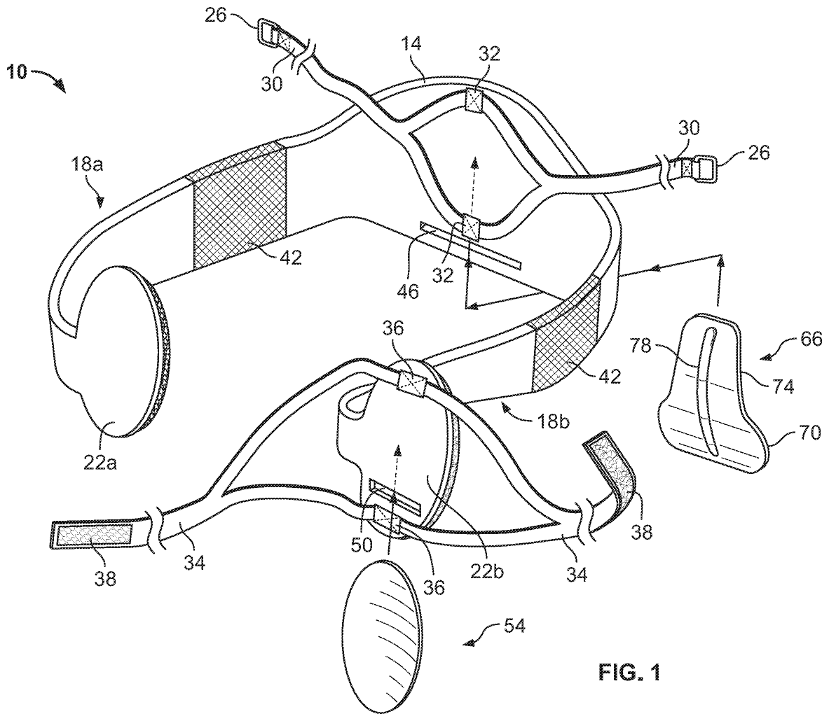

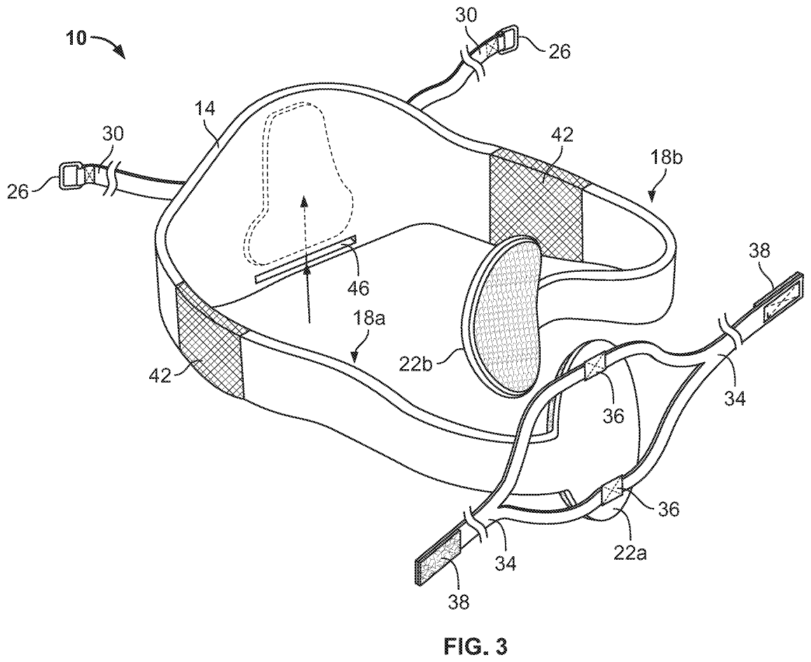

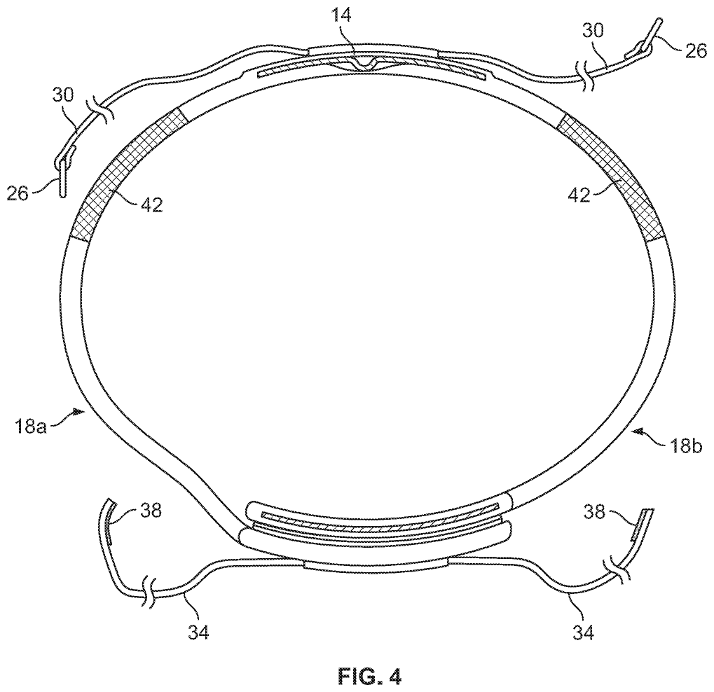

A belt 10 as illustrated in FIG. 1 includes a posterior panel 14 for application to a posterior side of a wearer's abdomen. A first wing 18a and second wing 18b extend from opposite lateral edges of the posterior panel 14. The first wing 18a and second wing 18b terminate in a first anterior panel 22a and second anterior panel 22b, respectively. The first anterior panel 22a and second anterior panel 22b are each adapted for placement on an anterior side of the wearer's abdomen. The wings 18a, 18b, each include an elastic segment 42 enabling a length of the wings 18a, 18b, and by extension a maximum distance between the anterior panels 22a, 22b and the posterior panel 14, to vary. The elastic segments 42 may be, for example, six inches long. In other examples, the length of elastic segments 42 may be more or less than six inches long. The first anterior panel 22a and second anterior panel 22b are securable to one another, such that they may prevent each other from falling out of place when the posterior panel 14 and anterior panels 22a, 22b are applied to their respective sides of the wearer's abdomen. As shown in FIGS. 3 and 4, the posterior panel 14 may be applied to the wearer's back, then the wings 18a, 18b may be stretched until the anterior panels 22a, 22b overlap one another in an intended placement over the wearer's stomach. When both anterior panels 22a, 22b are in place, they may be secured to one another to hold the belt 10 to the wearer.

In some examples, the anterior panels 22a, 22b are mutually adhesible to enable the anterior panels 22a, 22b to be secured to one another. For example, an outward facing side of one of the anterior panels 22a, 22b and an interior facing side of the other may have complementary hook-and-loop patches. In other examples, the outward facing side of one of the anterior panels 22a, 22b and an interior facing side of the other may have other fastening means such as clips, adhesives, buttons, and clasps.

The belt 10 of the illustrated example is of a modular nature, and is configured to accept rigid plates (anterior plate 54 and posterior plate 66 in FIG. 1) to facilitate provision of hydrostatic lift in the wearer's abdomen. To that end, the posterior panel 14 includes a posterior panel slot 46 leading into a pocket within the posterior panel 14. Similarly, the second anterior panel 22b of the example illustrated in FIG. 1 includes an anterior panel slot 50 leading into a pocket within the second anterior panel 22b. However, in various alternative arrangements, either or both of the first anterior panel 22a and second anterior panel 22b may include the anterior panel slot 50 and corresponding pocket. The slots 46, 50 are reversibly closable, such as by hook-and-loop or zipper lining Though illustrated at respective anterior edges of the posterior panel 14 and second anterior panel 22b, the slots 46, 50 may be located at any portion of their respective panels in alternative arrangements.

Two first straps 30 both have Y shapes, with first and second ends of the respective Y shapes meeting at first strap anchor points 32 and third ends of the respective Y shapes connecting to rings 26. Second straps 38 similarly both have Y shapes, with first and second ends of the respective Y shapes meeting at second strap anchor points 36. Third ends of the respective Y shapes of the second straps 34 are free, and may therefore be looped through the rings 26. The free ends of the second straps 34 include adhesible patches 38, such as hook or loop patches, enabling the free ends of the second straps 34 to adhere to themselves. The free ends of the second straps 34 can be looped through the rings 26, pulled tight, and then adhered to themselves to secure the tightening. Tightening of the straps 30, 34, lessens the effective combined length of either pair of mutually secured straps, and therefore reduces the maximum possible distance between the anchor points on the two panels to which the straps 30, 34 are anchored.

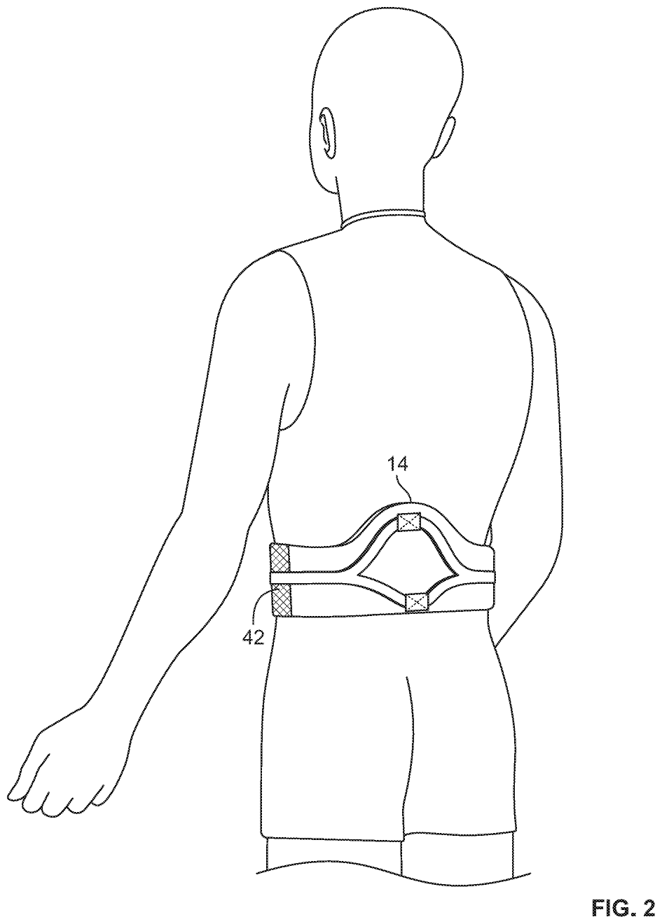

The first strap anchor points 32 anchor to an outer side of the posterior panel 14, and the second strap anchor points 36 anchor to an outer side of the second anterior panel 22b. As shown in FIG. 2, the anchor points 32, 36 are located on outer sides of their respective panels 14, 22b if the belt 10 is worn as intended. Thus, the straps 30, 34 extend around the outside of the belt 10 when the belt 10 is worn as intended. The belt 10 may, of course, be worn any way that the wearer wishes, but the intended arrangement shown in FIG. 2 enables the wearer to easily connect and tighten the straps 30, 34 when the panels 14, 22a, 22b are in place. The positioning of the straps 30, 34 on opposite panels 14, 22b enables the straps 30, 34 to cooperate to tighten the belt 10 about the wearer's abdomen. The elastic segments 42 may therefore have relatively low elastic moduli, because the straps 30, 34 enable tightening of the belt 10 irrespective of tension on the wings 18a, 18b. Moreover, the illustrated placement of the anchor points 32, 36 distributes the load on the straps 30, 34 across the wearer's abdomen. One of the first strap anchor points 32 is anchored at a relatively superior position near a superior edge of the posterior panel 14, and the other first strap anchor point 32 is anchored at a relatively inferior position near an inferior edge of the posterior panel 14. Similarly, one of the second strap anchor points 32 is anchored at a relatively superior position near a superior edge of the second anterior panel 22b, and the other second strap anchor point 32 is anchored at a relatively inferior position near an inferior edge of the second anterior panel 22b. Tension on the straps 30, 34 will thus be broadly distributed on the wearer's abdomen, particularly if the rigid plates are within the respective pockets in the posterior panel 14 and second anterior panel 22b.

In alternative arrangements, features of the belt 10 are varied from what is illustrated in FIG. 1. For example, as shown in FIG. 3, the second straps 34 may be anchored to the first anterior panel 22a, which does not include the pocket for the rigid plate. In such arrangements, whichever anterior panel 22a or 22b to which the second straps 34 are anchored may be configured for laying over the anterior panel 22a or 22b having the pocket as shown in FIG. 4, such that tension on the straps 30, 34 will be distributed first onto the rigid plate, then the wearer's abdomen. In further alternatives, one or both of the rings 26 may be anchored to either of the anterior panels 22a, 22b instead of the posterior panel 14, with a corresponding free-ended strap being anchored to the posterior panel 14. Moreover, one or both of the rings 26 may be any other feature suitable for securing one strap to another, including, for example, a loop, buckle, or another free end for tying to the opposite strap. The second straps 34 may end in a variety of other features, such clips, carabiners, or hooks for securing either to the rings 26 or back onto holes, hooks, or loops elsewhere on the second straps 34. In yet further alternatives, the first straps 30 do not meet at the first strap anchor points 32, and the second straps 34 do not meet at the second strap anchor points 36. Instead, the straps 30, 34 anchored to either panel are anchored at mutually laterally offset points, so the tension on the straps 30, 34 is distributed both vertically and horizontally on the respective panels.

The above described tensioning of the straps 30, 34, with the forces distributed on their respective panels, serve to restrict movement of the wearer's abdomen and create a hydrostatic lift to the lumbar structure. The belt 10 is even better able to distribute force, restrict movement, and provide hydrostatic lift when the aforementioned rigid plates are in their respective pockets.

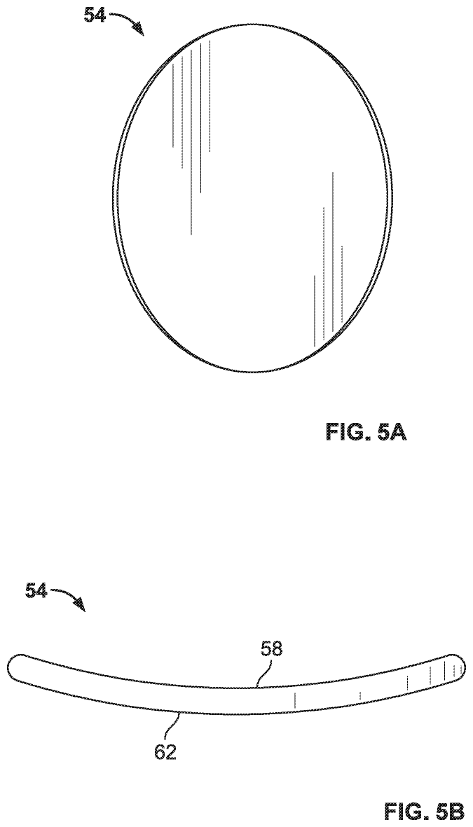

The anterior plate 54 is illustrated in FIGS. 5A and 5B. The anterior plate 54 has an oval shape, with a width to height ratio from the face-on perspective of FIG. 5A being 2:3. In some examples, the anterior plate 54 may have dimensions of 4″ by 6″, though the dimensions may vary in other sizes of the belt 10. From a top perspective view of FIG. 5B, the plate 54 has an arcuate shape subtending a 30° angle. It is noted that the figures are not to scale with respect to the measurements or angles described here. However, a variety of proportions and angles are contemplated, including those shown in the figures. The belt 10 and anterior plate 54 may be positioned such that the inferior end of the anterior plate 54 sits at or just above the wearer's pubic bone.

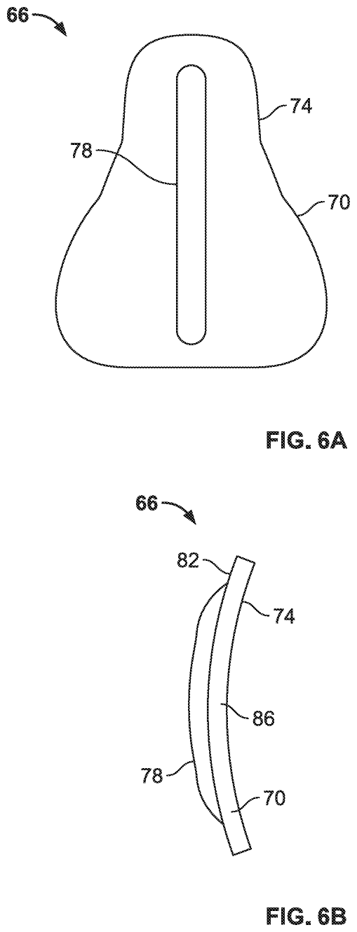

A posterior plate 66 is illustrated in FIGS. 6A and 6B. The posterior plate 66 includes a lumbar portion 70 that tapers laterally inward to a relatively laterally narrow thoracic portion 74. The lumbar portion 70 being laterally wider than the thoracic portion 74 enables the lumbar portion 70 to extend across a posterior side of the wearer's obliques. A rib 78 extends longitudinally along the lumbar portion 70 and thoracic portion 78. In the illustrated example, a ratio of a height of the posterior plate 66 to the lateral width of the lumbar portion 70 is 5:6, and a length of the rib 78 equals the width of the lumbar portion 70. In other examples, different ratios of height of the posterior plate 66 to the lateral with of the lumbar portion 70 are contemplated. In some examples, the posterior plate 66 may have a height of 6″ and the lumbar portion 70 may have a width of 5″. In other example, the posterior plate 66 may have a height more of less than 6″ and the lumbar portion 70 may have a width more or less than 5″.

Referring specifically to FIG. 6B, the posterior plate 66 has an arcuate shape subtending a 45° degree angle. An inner side 82, adapted to face the wearer's abdomen, is convex, while an opposed outer side 86 in concave. The rib 78 protrudes from the inner side 82 to extend between the wearer's spinal erectors and press on the wearer's spine.

The curvatures of the anterior plate 54 and posterior plate 66 cooperate to further facilitate creation of hydrostatic lift in the wearer's abdomen. The curve of the posterior plate 66 transfers some of the anterior-posterior load to superior and inferior directions on the wearer. The curve of the anterior plate 54, meanwhile, confines the abdomen laterally, causing lateral pressure within the abdomen to act in superior and inferior directions as well.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A lumbar support belt comprising:
a posterior panel;
a first anterior panel coupled to the posterior panel by a first wing extending from the posterior panel;
a second anterior panel coupled to the posterior panel by a second wing extending from the posterior panel;
first and second attachment portions attached to the posterior panel by a first posterior strap and second posterior strap, respectively, the first posterior strap and second posterior strap each being anchored to the posterior panel at two points; and
first and second anterior straps each anchored to either one of the first and second anterior panel at two points and securable to the first and second attachment portions.

2. The belt of claim 1, wherein the first and second anterior straps are each anchored to the first anterior panel at a relatively superior point and a relatively inferior point.

3. The belt of claim 1, wherein the first and second posterior straps are each anchored to the posterior panel at a relatively superior point and a relatively inferior point.

4. The belt of claim 1, wherein the first anterior strap and the second anterior strap each have a Y shape, with first and second ends being anchored to the respective one of the first anterior panel and second anterior panel and a third end being free.

5. The belt of claim 4, wherein the first posterior strap and the second posterior strap each have a Y shape, with first and second ends being anchored to the posterior panel and a third end being free, with the respective one of the first attachment portion and the second attachment portion being at the third end.

6. The belt of claim 1, wherein the first wing and the second wing each include an elastic segment enabling elongation of the respective one of the first wing and the second wing between the posterior panel and the respective one of the first anterior panel and second anterior panel.

7. The belt of claim 1, including a posterior plate receivable in a pocket in the posterior panel, the posterior plate including a lumbar portion and a thoracic portion that is adjacent to the lumbar portion in a longitudinal direction and narrower than the lumbar portion in a lateral direction that is perpendicular to the longitudinal direction.

8. The belt of claim 7, wherein the posterior plate includes a concave side, a convex side opposite the concave side, and a rib extending longitudinally along the convex side.

9. The belt of claim 1, wherein the first and second anterior straps are each securable to the respective first and second attachment portions at a user selectable position along the respective one of the first anterior strap and second anterior strap.

10. The belt of claim 1, wherein the first and second anterior panel are mutually adhesible.

11. The belt of claim 1, wherein the first and second attachment portion are a first and second ring secured to an end of the respective first or second posterior strap.

12. A lumbar support belt comprising:
a posterior panel;
first and second posterior straps each anchored to the posterior panel at two points;
an anterior panel; and
first and second anterior straps each anchored to the anterior panel at two points, the first and second anterior straps each being securable to a respective one of the first and second posterior straps such that, when secured, a combined length of the first anterior strap and first posterior strap is user selectable and a combined length of the second anterior strap and the second posterior strap is user selectable.

13. The belt of claim 12, wherein the anterior panel is coupled to the posterior panel by a wing extending from the posterior panel.

14. The belt of claim 12, including a connecting panel adhesible to the anterior panel, the connecting panel being coupled to the posterior panel by a wing extending from the posterior panel.

15. The belt of claim 12, wherein the first and second anterior straps are each anchored to the first anterior panel at a relatively superior point and a relatively inferior point.

16. The belt of claim 12, wherein the first and second posterior straps are each anchored to the posterior panel at a relatively superior point and a relatively inferior point.

17. A method of providing hydrostatic lift including:
placing a posterior panel against a posterior of a wearer's abdomen;
placing a first anterior panel against an anterior of the wearer's abdomen; and
tightening the posterior panel and first anterior panel against the wearer's abdomen by adjusting a connection between a first posterior strap anchored at two points to the posterior panel and a first anterior strap anchored at two points to the anterior panel and a connection between a second posterior strap anchored at two points to the posterior panel and a second anterior strap anchored at two points to the anterior panel.

18. The method of claim 17, comprising placing a second anterior panel against the anterior of the wearer's abdomen before tightening the posterior panel and first anterior panel against the wearer's abdomen.

19. The method of claim 18, wherein placing the first anterior panel against the anterior of the wearer's abdomen includes placing the first anterior panel over the second anterior panel and establishing an adhesive connection between the first and second anterior panels.

20. The method of claim 17, wherein adjusting the connection between the first posterior strap and the first anterior strap and adjusting the connection between the second posterior strap and second anterior strap include either pulling the first and second anterior straps through rings connected to the first and second posterior straps, or pulling the first and second posterior straps through rings connected to the first and second anterior straps.
